(19) United States
(12) Patent Application Publication
(10) Pub. No.: US 2020/0164290 A1
(43) Pub. Date: May 28, 2020

(54) ROTARY VACUUM FILTER, METHOD, AND USE

(71) Applicant: SABIC GLOBAL TECHNOLOGIES B.V., Bergen op Zoom (NL)

(72) Inventors: Aaron David Bojarski, Cartagena (ES); Manuel Iglesias Moya, Cartagena (ES); Juan Francisco Ceron Munoz, Cartagena (ES); Gines Cervantes Munoz, Carretera Cartegena (ES)

(21) Appl. No.: 16/615,192

(22) PCT Filed: Jun. 7, 2018
(86) PCT No.: PCT/IB2018/054128

§ 371 Date: Nov. 20, 2019

(30) Foreign Application Priority Data
Jun. 7, 2017 (EP) 17382350.1

(51) Int. Cl. B01D 33/073; B01D 33/067; B01D 33/76; B01D 33/00; B01D 33/46; B01D 33/72

(52) U.S. Cl. CPC B01D 33/073; B01D 33/067; B01D 33/76; B01D 33/72; B01D 33/466; B01D 2201/282; B01D 33/0064

(57) ABSTRACT

A rotary vacuum filter for separation of bisphenol A-phenol adduct crystals from a crystallization liquor is provided. The rotary vacuum filter includes a filter drum with a perforated sector on a lateral surface of the filter drum; a non-polymeric filter cloth attached to the filter drum and covering said perforated sector; and a vacuum pump in fluid communication with an interior surface of the perforated sector. Methods and uses of thereof are also provided.

### BACKGROUND

Bisphenol A is commercially produced by the condensation reaction of acetone and two equivalents of phenol in the presence of a catalyst such as an ion-exchange resin. In order to separate the formed bisphenol A from the product mixture, a crystallization step in a crystallization unit is often performed. The resulting bisphenol A-phenol adduct crystals can be separated from the crystallization liquor using a rotary vacuum filter.

DE 199 61 521 to Rainer et al. is directed to a method for the isolation and purification of bis-(4-hydroxyaryl)-alkane/phenol adducts from the acid-catalyzed reaction of ketones with phenols involves separating the crystallized adduct from the mother liquor by continuous filtration in a rotary vacuum filter with several filter cells, washing the crystals and removing the washings by suction.

WO 02-055175 to Daniel is directed to a self-cleaning drum filter. The self-cleaning drum filter comprises a pressure vessel containing a cylindrical filtering drum with a filtering mat and means for removal of the cake collected on the outside of the filtering mat. The means for removal consists of a scanner plate juxtaposed to the inside wall of the pressure vessel. The scanner plate having, along its length, a through-slit centrally placed that provides a passage between the inner and outer faces of said scanner plate, with the outer face being provided, along the length of said slit (22), with a trough (21) which forms, together with the inner wall of said pressure vessel, a channel for collecting the flushing liquid during the cake-removal cleaning operation.

An improved rotary vacuum filter unit for the production of bisphenol A would be desirable.

### BRIEF DESCRIPTION

A rotary vacuum filter for separation of bisphenol A-phenol adduct crystals from a crystallization liquor can include a filter drum with a perforated sector on a lateral surface of the filter drum, a non-polymeric filter cloth attached to the filter drum and covering said perforated sector, and a vacuum pump in fluid communication with an interior surface of the perforated sector.

A filter unit can include the above-described rotary vacuum filter and a scraper comprising a scraper section proximate to said filter cloth for removing said bisphenol A-phenol adduct crystals from said filter cloth.

A method for separating bisphenol A-phenol adduct crystals from a crystallization liquor can include feeding a crystallized stream comprising the bisphenol A-phenol adduct crystals and the crystallization liquor to the above-described rotary vacuum filter or the above-described filter unit and separating the bisphenol A-phenol adduct crystals from the crystallization liquor to form a filter cake on said filter cloth and a crystallization liquor stream in said interior surface of said perforated sector.

The above described and other features are exemplified by the following figures, detailed description, examples, and claims.

### BRIEF DESCRIPTION OF THE DRAWINGS

The following figures are exemplary embodiments wherein the like elements are numbered alike.

FIG. 1 is an illustration of an embodiment of a bisphenol A production facility.

FIG. 2 is an illustration of an embodiment of a filter unit.

FIG. 3 is an illustration of an embodiment of a filter drum assembly.

FIG. 4 is an illustration of cross-section A-A of the filter drum assembly illustrated in FIG. 3 illustrating a lateral groove with an insert and a connector.

FIG. 5 is an illustration of a cross-section of another filter drum assembly illustrating a lateral groove with an insert.

FIG. 6 is an illustration of a cross-section of yet another filter drum assembly illustrating a lateral groove with a spring.

FIG. 7 is an illustration of a cross-section of yet another filter drum assembly illustrating a lateral groove with a metallic piece and a screw.

FIG. 8 is an illustration of a top view of metallic piece 470, screws 480, and filter cloths 326, 328 from the filter drum assembly illustrated in FIG. 7.

FIG. 9A is a graphical illustration of the filtrate weight change as a function of time for Examples 6 and 9, and Comparative Examples 1A, 1B, and 2.

FIG. 9B is a graphical illustration of the filtrate weight change as a function of time for Examples 1, 3-5, and 10.

FIG. 10A is a graphical illustration of the change of the filtrate weight over the change in time as a function of time for Examples 6 and 9, and Comparative Examples 1A, 1B, and 2.

FIG. 10B is a graphical illustration of the change of the filtrate weight over the change in time as a function of time for Examples 1, 3-5 and 10.

FIG. 11A is a picture of the tensile strength test setup used in Examples 14-21.

FIG. 11B is an illustration of the tensile strength test setup used in Examples 14-21.

FIG. 12 is an illustration of the parameters used in the fitting pressure calculations for Examples 14-21.

The above described and other features are exemplified by the detailed description, claims, and examples.

### DETAILED DESCRIPTION

Bisphenol A (BPA) production plants can comprise a filter unit including a rotary vacuum filter that can facilitate separation of the formed BPA-phenol adduct crystals from the crystallization liquor (i.e., mother liquor). It is noted that “adduct” as used herein refers to the physical association of bisphenol A and phenol (e.g., one mole of bisphenol A and one mole of phenol can crystallize together to form a 1:1 molar ratio of bisphenol A /phenol adduct). The BPA-phenol adduct crystals can be needle-shaped. A dimension (e.g., diameter, width, or length) of the BPA-phenol adduct crystals can be 80 micrometers to 200 micrometers. The rotary vacuum filter can include a filter cloth attached to a filter drum. The filter drum rotates over an agitated pan that maintains the BPA-phenol adduct crystals suspended in solution. Vacuum from inside of the filter drum facilitates formation of a filter cake of the BPA-phenol adduct crystal to form on the filter cloth by pulling crystallization liquor into the filter drum. A scraper is held proximate to (e.g., millimeters (mm) away from) the filter cloth for filter cake detachment and recovery.

The rotary vacuum filter can be subject to frequent shutdowns in order to replace worn, damaged, or detached polymeric filter cloths. For instance, the polymeric filter cloth can be damaged by the scraper, by the parts for attaching the polymeric filter cloth to the filter drum, or by abrasion between polymeric filter cloth layers. In addition, the polymeric filter cloth can be loosen from fitting grooves into which they are introduced for attachment to the filter drum. These issues can result in shutdown of the rotary vacuum filter, replacement of the polymeric filter cloth, and knife realignment, which can take amounts of time (e.g., approximately 6 days from shutting down to start up) and reduce productivity.

It was surprisingly found that modification of one or more of the following parameters: (1) filter cloth material, (2) filter cloth layout, (3) fastening (i.e., attaching) method, and (4) scraper material as described in the present disclosure, renders an improved rotary vacuum filter for separation of BPA-phenol adduct crystals from a crystallization liquor. For instance, an increase in the mean time between failures of the rotary vacuum filter and a decrease in the time for repairs can be achieved. An increase in the mean time between failures can also result in improvement of other production based metrics such as lower mass of product lost, lower phenol usages due to lower BPA in the feed to reactors, lower nitrogen usages, lower steam usages due to less washing of the rotary vacuum filter, and lower amounts of phenol in the dehydration area.

As used herein the term “mean time between failures” refers to the period of time between starting the rotary vacuum filtration process after a shutdown to the next shutdown of the rotary vacuum filter. As used herein the term “failure” refers to at least one of an amount of bisphenol A-phenol adduct crystals present in the crystallization liquor of equal to or greater than 25 weight %, based on the total weight of the crystallization liquor; and a fluctuation in the vacuum pressure with the same frequency as the filter drum rotations per minute (e.g., fluctuations in vacuum pressure occurring the same number of times per minute as the filter drum rotations per minute, which can indicate damage, e.g., holes, in the filter cloth) The failures can be due to wear or damage to the filter cloths, wear or damage to the scraper, or loosening of the filter cloths from the filter drum.

A rotary vacuum filter for separation of bisphenol A-phenol adduct crystals from a crystallization liquor was developed that surprisingly increases the mean time between failures. The rotary vacuum filter includes a filter drum with a perforated sector on a lateral surface of the filter drum, a non-polymeric filter cloth attached to the filter drum and covering the perforated sector (i.e., a filter drum assembly), and a vacuum pumps in fluid communication with the perforated sectors.

A rotary vacuum filter can include more than 1, or more than 2 filter drum assemblies. A filter drum can be made up of one or more perforated sectors on a lateral side of the filter drum. Desirably, a filter drum can include 6 or more perforated sectors, 10 or more perforated sectors, or 20 or more perforated sectors. The lateral side of the filter drum can have one or more annular grooves and one or more lateral grooves adjacent to the perforated sections.

The grooves can have an inside width that is greater than a surface width. For example, the groove can have a width at adjacent the surface of the drum (W₁), and a width below the surface of the drum (in the groove) (W₂), wherein W₂>W₁; preferably, W₂≥1.1W₁; or W₂≥1.2W₁. With an inside width that is greater than the surface width, when pressure is applied to the insert (e.g., a thermoplastic elastomeric insert), the insert deforms, spreading within the groove, and more firmly anchoring the ends of the filter cloth in the groove.

Perforated sections can be covered with one or more filter cloths attached to the filter drum. Desirably, a filter drum assembly includes 2 or more filter cloths 224, 6 or more filter cloths, or 10 more filter cloths, or 20 or more filter cloths. For instance, each perforated sector can be covered by one filter cloth.

As used herein, “non-polymeric filter cloth” or “filter cloth” refers to any material through which a fluid can be filtered and comprising a material other than a thermoplastic polymer. The non-polymeric filter cloth can be metallic. For instance, the non-polymeric filter cloth can be stainless steel.

An attaching piece for attaching the filter cloth to the filter drum of a rotary vacuum filter was developed that surprisingly increases the mean time between failures. The attaching piece can be an insert in a groove adjacent to the perforated sectors, wherein at least a portion of the filter cloth is fixed in the groove by the insert abutting the portion of the filter cloth opposite an interior surface of the groove. The insert can be a thermoplastic elastomer or a metallic material. The insert can be a cord, such as a thermoplastic elastomer cord, a spring, a metallic cable, or a metallic piece. For instance, several metallic bars can be inserted end-to-end into a groove adjacent to the perforated sectors. The length of the metallic pieces can be in the range of 60 to 200 millimeters.

More than 1, or more than 2 inserts can be present in an annular groove or a lateral groove.

An attaching piece can be a connector that further assists in maintaining the insert in the groove, for example that increases the compression (e.g., fitting pressure) of the filter cloth against the interior surface of the grooves. The connector can be a bolting piece (e.g., a bolt, screw, rivot, bar, and so forth). One connector (e.g., bolting piece), more than 1, or more than 2, or more than 5, or more than 10 can be present in a groove or an attaching piece. For example, the insert can be compressed into the groove with multiple fasteners (e.g., connectors) that compress the insert.

The attaching pieces ensure that there is little or no relative movement between the filter drum and the filter cloth and allow for ease of removal to reduce the time for replacement of filter cloths.

The non-polymeric filter cloth can be woven or nonwoven. For instance, the non-polymeric filter cloth can be woven in a Dutch weave, a reverse Dutch weave, a heddle atlas weave, or a square weave. The non-polymeric filter cloth can be sintered fibers.

A pore size of the non-polymeric filter cloth can be 50 micrometers to 350 micrometers, or 150 micrometers to 250 micrometers.

A thickness of the non-polymeric filter cloth can be equal to or less than 1 millimeter (mm), e.g., 0.1 mm to 1 mm, preferably 0.25 mm to 0.75 mm. This thickness allows for bending the filter cloth and introducing the cloth into a groove of the filter drum for fastening of the non-polymeric filter cloth.

An average weight change of the crystallization liquor during separation of the bisphenol A-phenol adduct crystals from the crystallization liquor by the filter cloth can be equal to or greater than 4.0 grams per second (g/s). This permeability of the non-polymeric filter cloth can provide a low cake wetness and a low level of impurities in the separated material.

The filter cloth can have a tensile strength and elastic limit to allow for the bending and fastening of the non-polymeric filter cloth to a groove in the filter drum. For instance, the non-polymeric filter cloth can have a tensile strength equal to or greater than 400 Newtons per centimeter (N/cm), or equal to or greater than 500 N/cm, or equal to or greater than 600 N/cm in the machine direction, the cross direction, or both. The tensile strength can be measured by the ISO 527 test method.

The non-polymeric filter cloth can each cover one sector or cover multiple sectors. Thus, if wear or damage (such as a hole) is detected in one sector, that piece of non-polymeric filter cloth can be removed while keeping the other filter cloths in place.

The non-polymeric filter cloth can be attached to the filter drum in a single layer to avoid abrasion between multiple layers.

One vacuum pump, more than one, or more than 2 vacuum pumps can be present in a rotary vacuum filter.

The mean time between failures of the non-polymeric filter cloth for separation of bisphenol A-phenol adduct crystals from a crystallization liquor can be greater than or equal to 500 days, or greater than or equal to 2 years, or greater than or equal to 3 years, or greater than or equal to 4 years, or greater than or equal to 5 years. The mean time between failures of the non-polymeric filter cloth for separation of bisphenol A-phenol adduct crystals from a crystallization liquor can be 500 days to 5 years, or 2 to 5 years.4

The mean time between failures of the rotary vacuum filter for separation of bisphenol A-phenol adduct crystals from a crystallization liquor can be greater than or equal to 500 days, or greater than or equal to 2 years, or greater than or equal to 3 years, or greater than or equal to 4 years, or greater than or equal to 5 years. The mean time between failures of the rotary vacuum filter for separation of bisphenol A-phenol adduct crystals from a crystallization liquor can be 500 days to 5 years, or 2 to 5 years.

A filter unit for separation of bisphenol A-phenol adduct crystals from a crystallization liquor was developed that surprisingly increases the mean time between failures. The filter unit includes the above-described rotary vacuum filter and a scraper comprising a scraper section proximate to the filter cloth for removing said bisphenol A-phenol adduct crystals from the filter cloth.

The scraper section can include a polymer selected from at least one of polyether ether ketone or polytetrafluoroethylene, preferably comprises both polyether ether ketone and polytetrafluoroethylene. The scraper section can include a polyether ether ketone. The scraper section can include polytetrafluoroethylene. The polymer can include a filler, such as at least one of glass fiber, including textile glass fibers such as E, A, C, ECR, R, S, D, NE glasses, or quartz. The glass fiber can be present in the polymer in an amount equal to or less than 30 wt. %.

A Mohs hardness of the scraper can be less than a Mohs hardness of the filter cloth. As the scraper is softer than the non-polymeric filter cloth, the scraper can wear faster than the non-polymeric filter cloth. Changing the scraper can take less time than changing the filter cloth, and thus, time for repairs can be reduced. In addition, a softer scraper can reduce damage to the filter cloth by the scraper itself.

One scraper, more than one scraper, or more than 2 scrapers or scraper sections can be present. Desirably, 1 to 5 scrapers can be present. When more than one scraper is used, the scrapers can be positioned back-to-back (e.g., to provide a double edge) or at various positions or angles proximate to the filter drum assembly.

The use of more than one scraper section, preferably 2 to 5 scraper sections, allows for ease of alignment of the scraper relative to the filter drum assembly, as each scraper section can be separately adjusted and aligned proximate to the filter drum assembly in order to remove the filter cake from the non-polymeric filter cloth. One or more scraper sections can be replaced when damaged to avoid replacing the whole scraper. Thus, the undamaged portions of the scraper can be further utilized and less material discarded or wasted. The scraper sections can be attached along the scraper length (and along the lateral surface of the filter drum) end-to-end adjacent to one another by bolting each section to a bar proximate to and traversing the lateral surface of the filter drum.

As used herein, “scraper” refers to any article that can be used to physically remove the filter cake from the filter cloth, such as a blade or a knife.

A method for separating bisphenol A-phenol adduct crystals from a crystallization liquor can include feeding a crystallized stream into the above-described rotary vacuum filter or the above-described filter unit, and separating the bisphenol A-phenol adduct crystals from the crystallization liquor to form a filter cake on said filter cloth and a crystallization liquor stream. With the presently disclosed filter a mean time between failures of the rotary vacuum filter is increased as compared to a unitary cloth. With the present filter, the mean time between failures can be equal to or greater than 500 days, e.g., greater than or equal to 18 months, greater than or equal to 24 months, or greater than or equal to 36 months.

The filter unit can comprise two or more filter units operating in different lines. For example, a crystallized stream can be split into two or more portions and each respective portion directed to a separate filter unit.

A portion of the crystallization liquor produced by the rotary vacuum filter can be combined with phenol and acetone before the mixed stream is fed into a bisphenol A reactor. An example of such an alternate process for recycling filtrate is described in U.S. Patent Application Publication 2013/0221837 A1, which is incorporated in its entirety herein by reference.

The product mixture used to form the crystallized stream can comprise one or more of 15 to 40 wt %, or 20 to 35 w t% of bisphenol A; 60 to 85 wt %, or 55 to 70 wt % of phenol; 5 to 15 wt % of byproduct; 0 to 5 wt %, or 0 to 3.5 wt % of water; and 0 to 8 wt %, or 0 to 1.5 wt % of acetone; all based on a total weight of the product mixture.

Use of the above-described rotary vacuum filter or the above-described filter unit for separating bisphenol A-phenol adduct crystals from a crystallization liquor is provided.

A more complete understanding of the components, processes, and apparatuses disclosed herein can be obtained by reference to the accompanying drawings. These figures (also referred to herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

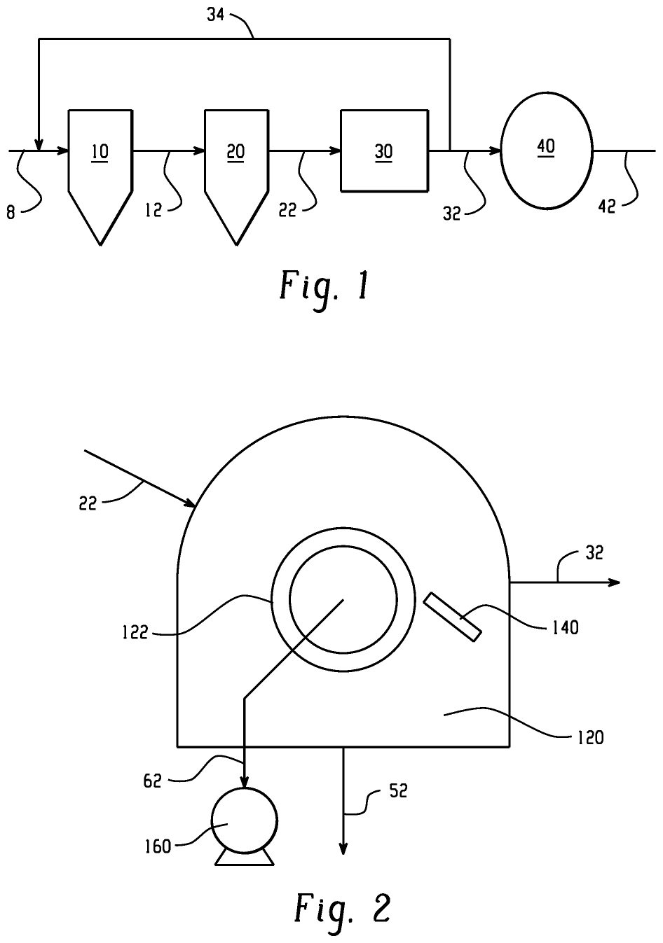

As illustrated in FIG. 1, a bisphenol A production facility can include reactor feed stream 8 directed to bisphenol A reactor 10 to form bisphenol A stream 12. Reactor feed stream 8 can comprise phenol, acetone, and optionally a promoter. Bisphenol A reactor 10 can be a fixed bed reactor comprising a catalyst. The phenol and acetone can be present in an amount of 5 to 15 moles of phenol per mole of acetone. Reactor feed stream 8 can comprise 75 to 95 weight percent (wt %) phenol and 1 to 8 wt % acetone. The phenol and acetone can be combined in a formulation tank located upstream of bisphenol A reactor 10. A portion of the crystallization liquor 34 from a downstream filtration process, such as at filter unit 30, can be combined with the phenol and acetone before the mixed stream is fed into bisphenol A reactor 10. Bisphenol A stream 12 can be removed from bisphenol A reactor 10.

Bisphenol A stream 12 comprises the product mixture from the bisphenol A reaction.

Bisphenol A stream 12 comprising the product mixture can be directed to crystallization unit 20 to form bisphenol A crystals comprising, for example, one or both of crystalline bisphenol A and an adduct of bisphenol A and phenol. The crystallization unit can comprise two or more crystallization units 20 operating in different lines. For example, the bisphenol A stream can be split into two or more portions and directing each respective portion to a separate crystallization unit. The crystals can be separated by removing the solid portion from the crystallization unit 20 comprising the crystals, for example, via filtration. To do so, crystallized stream 22 can be directed to filter unit 30 to form a crystallization liquor and a filter cake.

As illustrated in FIG. 2, crystallized stream 22 can be fed to rotary vacuum filter 120 of a filter unit. Rotary vacuum filter 120 can include filter drum assembly 122 in fluid communication with vacuum pump 160, which provides vacuum 62 to filter drum assembly 122. Proximate filter drum assembly 122 is scraper 140. As filter cake stream 32 is formed on the filter drum assembly, crystallization liquor 52 is formed by the vacuum in filter drum assembly 122.

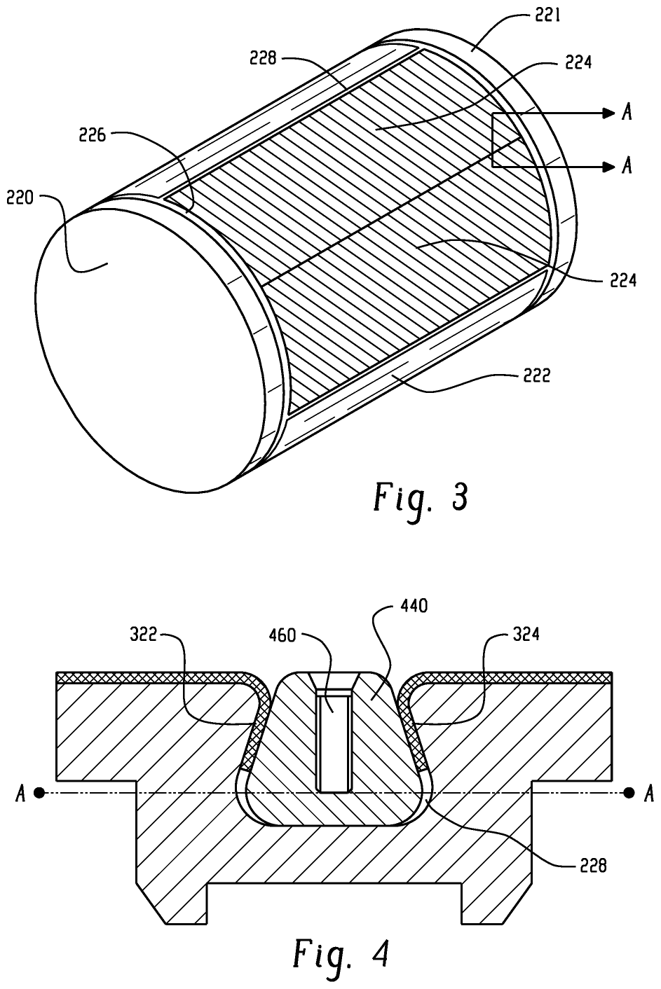

As illustrated in FIG. 3, filter drum 220 can be made up of a plurality of perforated sectors 222 on lateral side 221 of filter drum 220. Lateral side 221 can have annular grooves 226 and lateral grooves 228 adjacent to perforated sections 222. Perforated sections 222 can be covered with filter cloths 224 attached to filter drum 220.

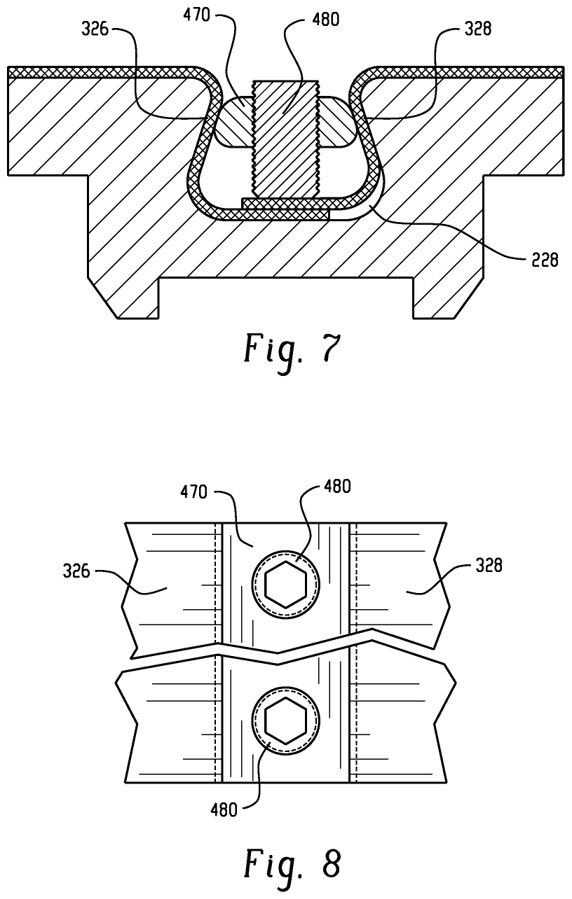

As illustrated in cross-section A-A of FIG. 4, a portion of first filter cloth 322 can be bent and introduced into lateral groove 228 on one side. A portion of second filter cloth 324 can be bent and introduced into lateral groove 228 on the opposite side. Insert 440 can be introduced into groove 228, abutting and compressing against portion of first filter cloth 322 and portion of second filter cloth 324 towards the sides of lateral groove 228. Connector (e.g., bolting piece) 460 can be introduced into a portion of insert 440 to apply further compression by insert 440 on portion of first filter cloth 322 and portion of second filter cloth 324. Bolting piece 460 can be a bolt, screw, rivet, bar, etc., (preferably a bolt or screw) introduced to expand insert 440 to increase the compression towards the sides of lateral groove 228. Preferably an end of the first filter cloth overlaps an end of the second filter cloth (see FIG. 7), e.g., such that the insert 440 further presses the ends of the filter cloths together, thereby further securing them in the groove.

Thus, in FIG. 3 filter cloths 224 are bent around their perimeters into the corresponding annular grooves 226 and lateral grooves 228 for attachment to filter drum 220 using inserts, bolting pieces, or both.

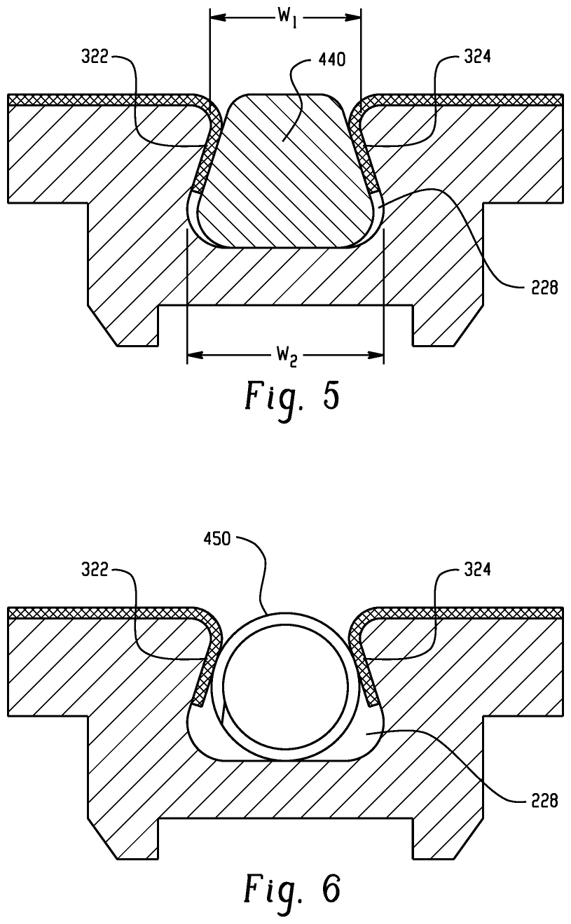

As illustrated in FIG. 5, an alternate filter drum assembly does not include bolting piece 460.

As illustrated in FIG. 6, another filter drum assembly includes spring 450 introduced into groove 228 instead of insert 440.

As illustrated in FIG. 7, still another filter drum assembly includes a metallic piece 470 in the shape of a bar instead of insert 440. As illustrated in FIG. 8, connector (e.g., a bolt, screw, rivet, bar, or the like), e.g., screws 480 can be inserted into metallic piece 470 along its length. When the screws 480 are screwed into metallic piece 470, the cross-sectional profile of metallic piece 470 increases the compress against portion of first cloth 326 and portion of second cloth 328, achieving a higher compression than the compression exerted by at least some other attaching pieces.

As illustrated in FIG. 1, filter cake stream 32 can be directed to melting unit 40 to form melted stream 42. The melting unit 40 can melt the crystals, for example, by heating the crystals at a temperature greater than the crystallization temperature. An additional amount of phenol can be added to the filter cake stream 32 to facilitate the melting of the crystals at a lower temperature. The melted stream can be further purified to produce a product bisphenol A. The product bisphenol A can be solidified, for example, in a flaking unit or a prilling tower (not shown in FIG. 1).

This disclosure is further illustrated by the following examples, which are non-limiting.

### EXAMPLES

Exemplary stainless steel filter cloths and their characteristics are summarized below in Table 1.

TABLE 1

| Sample | 1 | 2 | 3 | 4 | 5 | 6 | 7 | 8 | 9 | 10 |
|---|---|---|---|---|---|---|---|---|---|---|
| Type | 30 warp | 24 warp | 150S | 200S | TELA ^{™} | 12 warp | 98 warp | 78 warp | 49 warp | |
| | x 150 | x 110 | | | 5110/140 | x 64 weft | x 98 weft | x 78 weft | x 49 weft | |
| | weft | weft | | | | | | | | |
| Weave | Plain | Plain | Reverse | Reverse | Heddle | Plain | Square | Square | Square | Sintered |
| | Dutch | Dutch | Plain | Plain | Atlas | Dutch | Weave | Weave | Weave | Fiber |
| | Weave | Weave | Dutch | Dutch | Weave | Weave | | | | |
| | | | Weave | Weave | | | | | | |
| Geometrical | 122 | 153 | 144 | 148 | 98 | 301 | 160 | 200 | 315 | 58 |
| Pore Size | | | | | | | | | | |
| (micrometers) | | | | | | | | | | |
| Tensile Strength | 423 | 756 | 1267 | 1260 | — | 750 | — | — | — | — |
| Warp (N/cm) | | | | | | | | | | |
| Tensile Strength | 750 | 948 | 1006 | 914 | — | 2620 | — | — | — | — |
| Weft (N/cm) | | | | | | | | | | |
| Thickness (mm) | 0.503 | 0.75 | 0.922 | 0.932 | 0.426 | 1.21 | 0.221 | 0.262 | 0.411 | — |
| Cloth weight | 1.53 | 2.59 | 3.11 | 3.09 | 1.28 | 4.1 | 0.49 | 0.59 | 0.96 | — |
| (kilogram per | | | | | | | | | | |
| meter squared | | | | | | | | | | |
| (kg/m²)) | | | | | | | | | | |

### Examples 1-7 and Comparative Examples A-B: Filtration Cloth Permeability

Seven filter cloths were tested for their filtration rate of para-para-bisphenol A-phenol (ppBPA-phenol) adduct crystals in phenol. In addition, two comparative filter cloths were tested. Comparative Example A was a polyether ether ketone (PEEK) filter cloth. Comparative Example B was two layers of polypropylene (PP) filter cloth. The properties of these filter clothes is summarized in Table 2.

TABLE 2

| | PEEK filter cloth | PP filter cloth | |
|---|---|---|---|
| Manufacturer | SEFAR | PURE AIR, INC. | |
| Material | (pre-shrunk) | 8.5 ounce, | 15 mil (0.381 |
| Style | | 12 mil (0.305 mm) | mm) |

The ppBPA-phenol adduct crystals were prepared by crystallizing 390 grams (g) of reactor effluent at 70° C. (with 1 wt % seeding) and cooled to 56° C. The slurry formed provides a filter cake approximately 10 centimeters (cm) thick when filtered with a laboratory pocket filter having a heated jacket and drain point. An overpressure of 0.05 bar (5 kiloPascals (kPa)) at 56° C. was used. The filtrate flow leaving the filter was measured every two seconds using a scale connected to an appropriate data acquisition software. Measurements on the filtered liquid filtrate (permeate) show that ppBPA content is approximately 12 to 13 weight percent for all the tested filtrates.

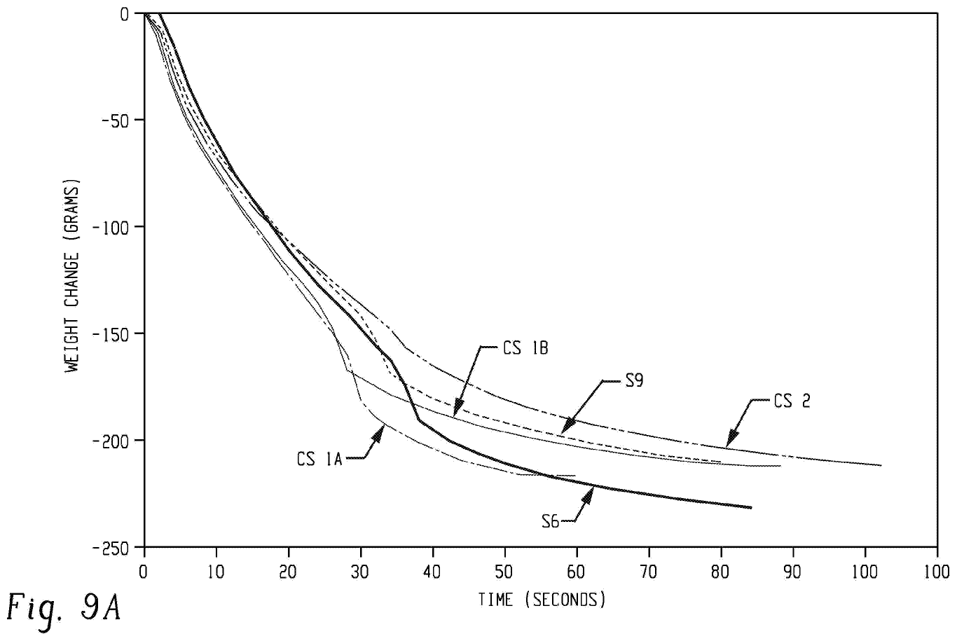

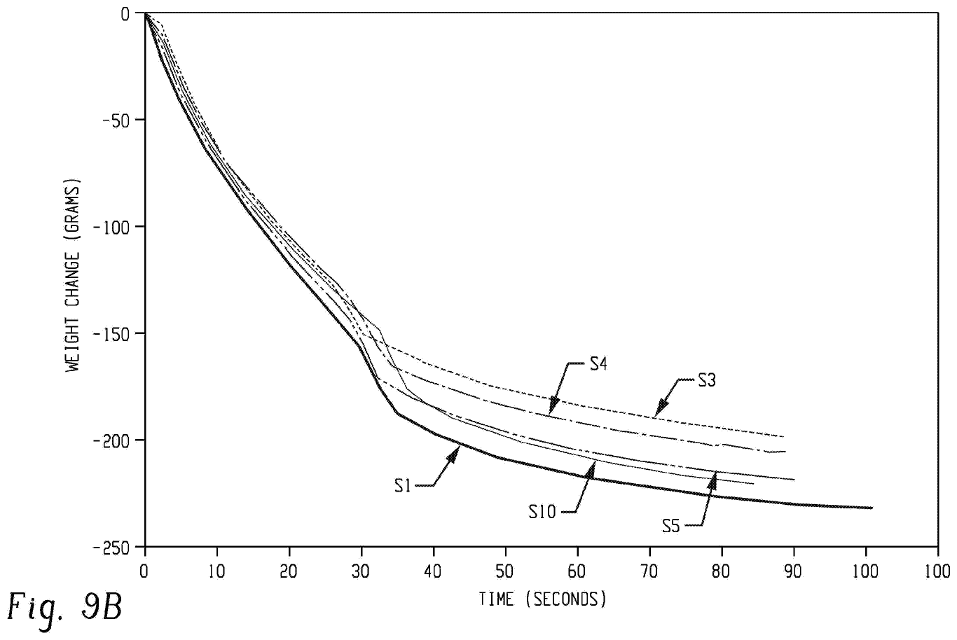

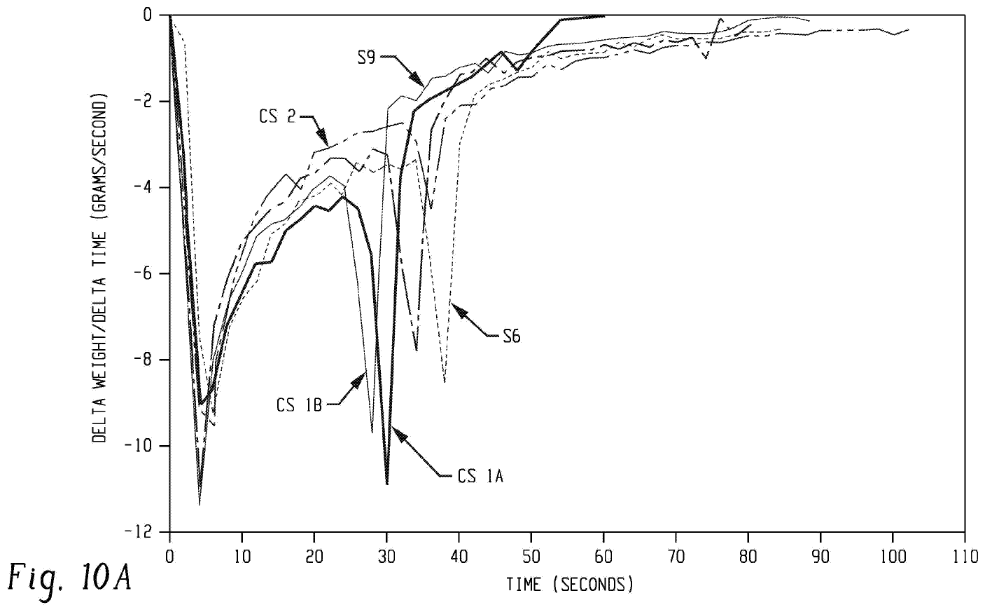

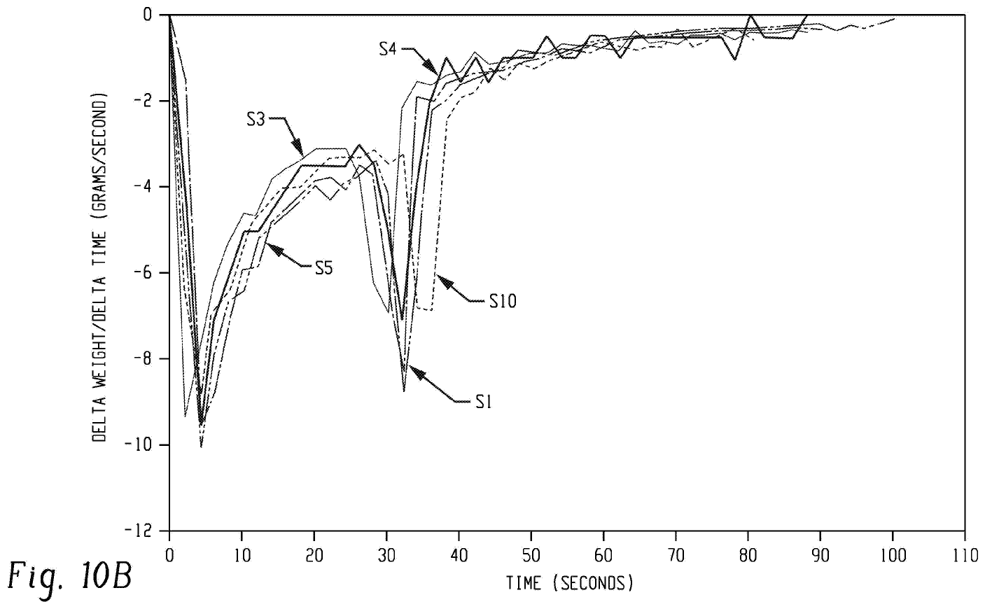

FIGS. 9 and 10 show a first phase (Phase 1) with a greater drop in filtrate weight over time followed by another phase (Phase 2) with a lower change in filtrate weight over time. Noticeably in FIG. 9 there is a dip in the data trend when Phase 1 ends and Phase 2 begins.

The time period of Phase 1, the average filtrate weight change and standard deviation in Phase 1 and Phase 2, and the concentration of ppBPA-phenol adduct crystals in the filtrate for the samples tested, is summarized in Table 3. As the cake generated is the same for each example, the filter cloth is the variable correlating to the different results. Thus, the average filtrate weight change provides a measure of the filtration performance.

TABLE 3

| | | | | | | Concentration of |
|---|---|---|---|---|---|---|
| | | | Phase 1 standard | | Phase 2 standard | ppBPA-phenol |
| | Phase 1 time | Phase 1 average | deviation of | Phase 2 average | deviation of | adduct crystals in |
| Sample | (seconds)¹ | weight drop (g/s) | weight drop (g/s) | weight drop (g/s) | weight drop (g/s) | filtrate (wt %) |
| Comparative | 30 | −5.55 | 1.35 | −1.33 | 0.95 | 12.9* |
| Sample A.1 | | | | | | |
| Comparative | 28 | −5.21 | 1.30 | −0.83 | 0.55 | |
| Sample A.2 | | | | | | |
| Comparative | 36 | −3.63 | 1.25 | −0.85 | 0.57 | 13.0 |
| Sample B | | | | | | |
| Sample 1 | 32 | −4.85 | 1.32 | −0.85 | 1.04 | 12.7 |
| Sample 5 | 32 | −5.05 | 1.56 | −0.82 | 0.48 | 13.0 |
| Sample 6 | 38 | −4.62 | 1.26 | −0.88 | 0.61 | 12.9 |
| Sample 9 | 34 | −4.42 | 1.27 | −0.91 | 0.55 | 13.0 |
| Sample 10 | 36 | −4.39 | 1.32 | −0.91 | 0.53 | 12.9 |
| Sample 3 | 30 | −4.50 | 1.46 | −0.82 | 0.44 | 13.3 |
| Sample 4 | 32 | −4.38 | 1.18 | −0.88 | 0.75 | 13.0 |

*Average for repetitions A.1 and A.2 of Comparative Sample A.
¹The time of Phase 1 until the start of Phase 2.

The summarized data in Table 3 shows that the suitability of these non-polymeric filter cloths for filtering ppBPA-phenol adduct crystals, as they provide an acceptable permeability and do not allow for a loss in fines into the filtrate. Conversely if a longer time period for Phase 1 was observed, coupled with very small flows on Phase 2 and Phase 1, the tested cloths would have been unacceptable for phenol-BPA adduct crystals filtering. Not to be bound by theory, the differences obtained in the flows for both phases are very small, thus cloths behave with respect to filtering in the same way, i.e., the cake is the most important factor (rather than the cloth itself).

### Examples 8-10: Chemical Resistance of Insert used to Fix the Filter Cloth to the Drum

Bisphenol A reactor effluent was contacted with samples of a SANTOPRENE™ 201-73 thermoplastic elastomer supplied by Eagle Burgmann Ibérica S.A, which can be used as the material for attaching piece inserts. The material was exposed to process fluids and temperature of 80° C. The insert material was measured and observed before and after exposure to determine if changes in length and weight occurred.

The insert was submerged in three different process stream solutions: (1) bisphenol A reactor effluent containing approximately 23 wt % of bisphenol A, 65 wt % of phenol, and 12 wt % of byproduct; all based on a total weight of the effluent;, (2) bisphenol A reactor effluent +5 wt % acetone; and (3) phenol and 5 wt % acetone. Mechanical stresses such as those associated with use on a rotary vacuum filter was induced on the insert by introducing bolting pieces into them. The cloths on top of the inserts were mechanically fixed. The original and resultant dimensions of the insert material are shown in Table 4.

TABLE 4

| | | | bisphenol A reactor | | | |
|---|---|---|---|---|---|---|
| | bisphenol A reactor | | effluent and 5 wt % | | Phenol and 5 wt % | |
| | effluent | | acetone | | acetone | |
| Time | Weight | | Weight | | Weight | Length |
| (hrs) | (g) | Length (mm) | (g) | Length (mm) | (g) | (mm) |
| 0 | 31.57 | 22.8 | 26.66 | 19.2 | 21.81 | 15.6 |
| 168 | 32.53 | 22.8 | 27.2 | 19.2 | 21.85 | 15.6 |
| 360 | 32.51 | 22.8 | 27.81 | 19.2 | 21.81 | 15.6 |
| 504 | 33.30 | 22.8 | 27.70 | 19.2 | 21.86 | 15.6 |

The results show almost no change in length and weight of the insert material. The inserts did not change in appearance nor were other visual defects observable after 500 hours of testing.

### Examples 11-21: Non-Polymeric Filter Cloth Fastening

In Examples 11-13, filter cloth Samples 1-3 were each fastened over three perforated sectors of a rotary vacuum filter drum using the attaching pieces illustrated in FIG. 4. The perforations were 0.5 centimeter circular perforations spaced 0.2 centimeters apart. Sample 1 could not be adjusted to conform to the curvature defects of the filter drum as well as Sample 2 and Sample 3. As such, Sample 1 was not in contact with the perforated sectors on some areas of the filter drum.

The filter cloths were covered with tape to simulate the effect of a filter cake on the filter cloths. As the tape has a lower porosity than a bisphenol A-phenol adduct filter cake, the measured cloth movement was greater than in operation. A pressure test was performed to check the cloth movement when the covered sector was pressurized, simulating the filter material behavior during a blowback phase.

A pressure regulator and manometer were used to maintain a constant pressure inside the sectors of 2 bars. The cloth displacement was measured by placing dial indicators on the filter cloth at three different sections: (1) close to the air intake; (2) at the center of the drum, and (3) in between sections (1) and section (2). At the beginning of every run the dial indicators were set to zero.

For all the filter cloths, movement was lowest at Section 1, which was closest to the annular strip and the air intake. For all the Samples, the location with the greatest amount of displacement (e.g., displacement of the filter cloth in the perpendicular direction with respect to the drum) was at the center of the drum length and in the center of the perforated sector, which is furthest away from the annular grooves and the lateral grooves. The filter cloth with the lowest movement was Sample 2 and filter cloth with the most movement was Sample 1.

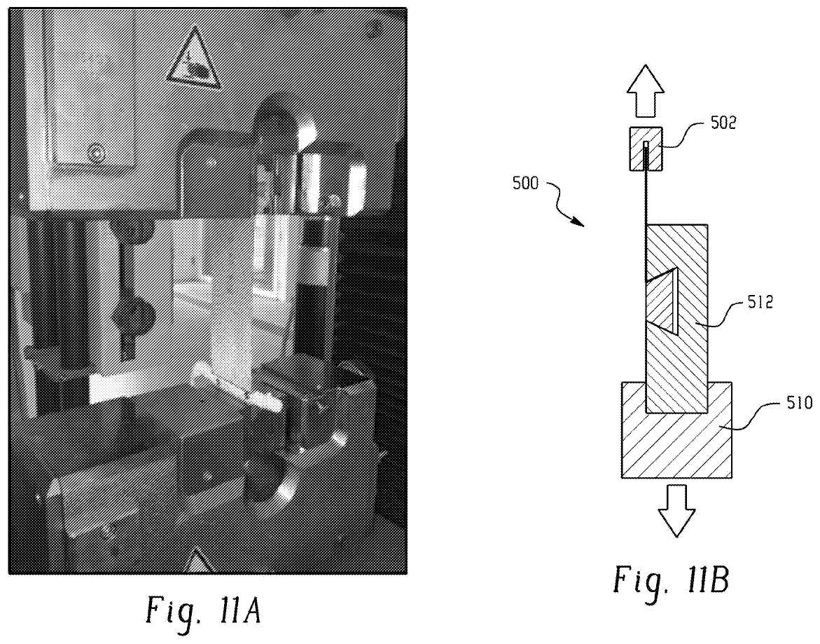

In Examples 14-21, the attaching pieces illustrated in FIG. 4 (Examples 14-20) and FIG. 7 (Example 21) were tested for fitting pressure (i.e., compression of the filter cloths by the attaching pieces) using the test setup shown in FIGS. 11A-B. As illustrated in FIG. 11B, each test assembly 500 includes test filter drum assembly 512 positioned on its end in holder 510. Holder 510 and filter cloth holder 502 exert a tensile force on test filter drum assembly 512.

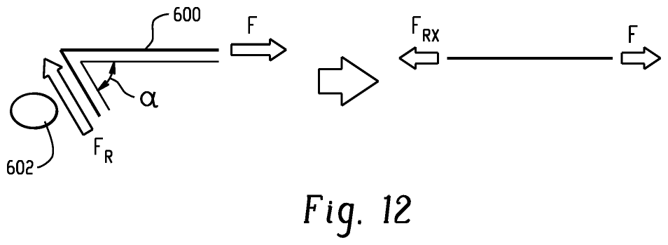

The resulting fitting pressure was calculated using Equation (1), Equation (2), and Equation (3). FIG. 12 illustrates the parameters used in Equations (1)-(3) in relation to filter cloth 600 and attaching piece 602. Tensile force upward (F) is equal to tensile force downward (F_{Rx}).

F_{R}=F_{Rx}/sin(α) Equation (1)

F_{R}=μ×N Equation (2)

τ=N/A_{d} Equation (3)

where F_{R} is the friction force between the attaching piece and the filter cloth, N is the normal force, μ is the coefficient of friction of the filter cloth, τ is the fitting pressure, and A_{d} is the contact area between the attaching piece and the filter cloth.

The effect of temperature and phenol contact was tested in Examples 14-20. The fitting pressures exerted by the attaching pieces illustrated in FIG. 4 and FIG. 7 were compared in Examples 14 and 21. The results of Examples 14-21 are shown on Table 5. No significant differences were seen between Examples 14-20. As it can be seen in the results, the fitting pressure for the attaching pieces of FIG. 7 was much higher than the fitting pressure of the attaching pieces of FIG. 4. Therefore, the fatigue stress over the filter cloth can be higher without the risk of loosening the filter cloth from the drum.

### Examples 22-29: Filter Unit Testing

Eight filter unit assemblies (including filter cloth, attaching pieces, and scraper) were tested for mean time between failures of a rotary vacuum filter for separation of bisphenol A-phenol adduct crystals from a crystallization liquor. Test of these examples was performed using a rotary vacuum filter to filter a bisphenol A reactor effluent as described in Examples 8-10 to produce approximately 50 tons/hour (13 kilograms per second (kg/s)) to 75 tons/hour (19 kg/s) of filtrate. These examples are summarized in Table 6.

In some examples the scraper was divided in different sections. For instance, the scraper in Example 22 was made up of 5 sections along its length (and along the lateral surface of the filter drum) and each section was attached end-to-end adjacent to one another by bolting each section to a bar proximate to and traversing the lateral surface of the filter drum. Having the several sections helped with aligning the scraper to the filter drum.

The following observations were made: In Examples 22, 23, 24, and 25 there was filter cloth wear and the formation of holes, resulting in replacement of whole filter cloth. Also, the stainless steel wire came loose and broke down in Example 23. In Example 26 attaching the single piece filter cloth (a single cloth with two ends) made holes in the filter cloth near the grooves. As there were holes in the filter cloth, adequate separation of bisphenol A-phenol adduct crystals from a crystallization liquor could not be achieved properly. In Example 27, there was scraper wear. Examples 28-29 did not exhibit any of the foregoing problems.

TABLE 5

| | Example 14 | Example 15 | Example 16 | Example 17 | Example 18 | Example 19 | Example 20 | Example 21 |
|---|---|---|---|---|---|---|---|---|
| Attaching | Cord insert | Cord insert | Cord insert | Cord insert | Cord insert | Cord insert | Cord insert | Metallic bar |
| Pieces | with bolts | with bolts | with bolts | with bolts | with bolts | with bolts | with bolts | with bolts |
| Test | Ambient | Tested after | Tested after | Tested after | Tested after | Tested after | Tested after | Ambient |
| conditions | temperature | 24 hours at | 150 hours at | 300 hours at | 150 hours in | 300 hours in | 600 hours in | temperature |
| | Test done | 100° C. | 45° C. | 45° C. | contact with | contact with | contact with | Test done |
| | after | | | | phenol at | phenol at | phenol at | after |
| | assembly | | | | 75° C. | 75° C. | 75° C. | assembly |
| Fitting | 3.6 | 3.2 | 3.3 | 3.3 | 2.8 | 3.0 | 3.2 | 53.2 |
| pressure | | | | | | | | |
| (kg/cm²) | | | | | | | | |

TABLE 6

| Item | Example 22 | Example 23 | Example 24 | Example 24.1 | Example 25 | Example 25.1 |
|---|---|---|---|---|---|---|
| Filter cloth | Single piece | Single piece | Single piece | Single piece | Single piece PP | Single piece PP |
| | polyether ether | polyether ether | polypropylene | polypropylene | (two layers) | (two layers) |
| | ketone | ketone | (two layers) | (two layers) | | |
| Attaching | Springs and | Springs and | Cord insert | Cord insert | Springs and | Springs and |
| Pieces | cord insert | cord insert + | | | cord insert | cord insert |
| | | stainless steel | | | | |
| | | wire | | | | |
| Scraper | Stainless steel | Stainless steel | Polytetra- | Polytetra- | Stainless steel | Stainless steel |
| | in 5 sections | in 5 sections | flouruoethylene + | fluoroethylene + | in 1 sections | in 5 sections |
| | | | glass fiber in | glass fiber in | | |
| | | | 1 section | 5 sections | | |
| Mean time | 2 months | 3 months | 6 months | 6 months | 15 months | 12 months |
| between | | | | | | |
| Failures | | | | | | |

| Item | Example 26 | Example 27 | Example 28 | Example 29 |
|---|---|---|---|---|
| Filter cloth | Single piece | One piece per perforated | One piece per perforated | One piece per perforated |
| | stainless steel | sector (20 pieces total), | sxecot (20 pieces total), | sector (20 pieces total), |
| | 316L | stainless steel 316L | stainless steel 316L | stainless steel 316L |
| Attaching | Springs and | Cord insert with bolts | Cord insert with bolts | Metallic bar with bolts |
| Pieces | cord insert | (FIG. 4) | (FIG. 4) | (FIG. 7) |
| Scraper | Polytetra- | Polytetrafluoroethylene + | Polyether ether ketone + | Polyether ether ketone + |
| | fluoroethylene + | glass fiber in 5 sections | polytetrafluoroethylene in 5 | polytetrafluoroethylene in 5 |
| | glass fiber in | | sections | sections |
| | 1 section | | | |
| Mean time | Not operable | 2 years (projected) | 4 years (projected) | 8 years (projected) |
| between | | | | |
| Failures | | | | |

Thus, the rotary vacuum filters and filter units described herein can be used to an increase in the mean time between failures of the rotary vacuum filter and a decrease in the time for repairs can be achieved.

This disclosure further encompasses at least the following aspects:

Aspect 1: A rotary vacuum filter for separation of bisphenol A-phenol adduct crystals from a crystallization liquor, the rotary vacuum filter comprising: a filter drum with a perforated sector on a lateral surface of the filter drum; a non-polymeric filter cloth attached to the filter drum and covering said perforated sector; and a vacuum pump in fluid communication with an interior surface of the perforated sector. The filter preferably further comprises a first attaching piece for attaching said filter cloth to the filter drum, wherein said first attaching piece comprises an insert positioned in a groove adjacent to said perforated sector, wherein at least a portion of said filter cloth is fixed in said groove by said insert abutting said portion of said filter cloth opposite an interior surface of said groove; and a second attaching piece comprising a connector (e.g., bolting piece) introduced into a portion of said insert;

Aspect 2: The rotary vacuum filter of Aspect 1, further comprising a first attaching piece for attaching said filter cloth to the filter drum.

Aspect 3: The rotary vacuum filter of Aspect 2, wherein said first attaching piece comprises an insert positioned in a groove adjacent to said perforated sector, wherein at least a portion of said filter cloth is fixed in said groove by said insert abutting said portion of said filter cloth opposite an interior surface of said groove.

Aspect 4: The rotary vacuum filter of Aspect 3, comprising 6 or more non-polymeric filter cloths; preferably 10 more non-polymeric filter cloths; or 20 or more non-polymeric filter cloths; preferably wherein each perforated sector is covered by one non-polymeric filter cloth.

Aspect 5: The rotary vacuum filter of any one or more of the preceding aspects, wherein the grooves can have an inside width (W₂) that is greater than a surface width (W₁), preferably W₂≥1.1W₁; or W₂≥1.2W₁.

Aspect 6: The rotary vacuum filter of any one or more of the preceding aspects, wherein, in the groove, an end of one non-polymeric filter cloth overlaps with an end of another non-polymeric filter cloth; preferably wherein the end of one non-polymeric filter cloth overlaps with the end of another non-polymeric filter cloth at a base of the groove and wherein the overlap is pressed into the drum by a connector (e.g., bolting piece; such as a bolt, screw, rivet, or bar; preferably a screw), e.g., that passes through the insert.

Aspect 7: The rotary vacuum filter of any one or more of Aspects 2-6, further comprising a second attaching piece comprising a connector (e.g., bolting piece; such as a bolt, screw, rivet, or bar; preferably a screw) introduced into a portion of said insert.

Aspect 8: The rotary vacuum filter of any one or more of the preceding aspects, wherein said filter cloth is metallic, preferably stainless steel.

Aspect 9: The rotary vacuum filter of any one or more of the preceding aspects, comprising two or more non-polymeric filter cloths and two or more perforated sectors, wherein said non-polymeric filter cloths are each covering to one of said perforated sectors.

Aspect 10: The rotary vacuum filter of any one or more of the preceding aspects, wherein said filter cloth is woven, preferably woven in a Dutch weave, a reverse Dutch weave, a heddle atlas weave, or a square weave.

Aspect 11: The rotary vacuum filter of any one or more of the preceding aspects, wherein a pore size of said filter cloth is 50 micrometers to 350 micrometers, or 150 micrometers to 250 micrometers.

Aspect 12: The rotary vacuum filter of any one or more of the preceding aspects, wherein a thickness of said filter cloth is equal to or less than 1 millimeter.

Aspect 13: The rotary vacuum filter of any one or more of the preceding aspects, wherein one layer of said filter cloth is covering said perforated sector.

Aspect 14: The rotary vacuum filter of any one or more of the preceding aspects, wherein a cloth weight of said filter cloth is equal to or greater than 0.40 kilograms per meter squared.

Aspect 15: A filter unit comprising: the rotary vacuum filter of any one or more of the preceding aspects; and a scraper comprising a scraper section proximate said filter cloth for removing said bisphenol A-phenol adduct crystals from said filter cloth.

Aspect 16: The filter unit of Aspect 15, wherein said scraper comprises at least one of polyether ether ketone and polytetrafluoroethylene; preferably comprises both polyether ether ketone and polytetrafluoroethylene.

Aspect 17: A method for separating bisphenol A-phenol adduct crystals from a crystallization liquor, the method comprising: feeding a crystallized stream comprising the bisphenol A-phenol adduct crystals and the crystallization liquor to the rotary vacuum filter of any one or more of Aspects 1 to 14 or the filter unit of any one or more of Aspects 15 to 16; and separating the bisphenol A-phenol adduct crystals from the crystallization liquor to form a filter cake on said filter cloth and a crystallization liquor stream in said interior surface of said perforated sector, preferably, a mean time between failures of the rotary vacuum filter is equal to or greater than 500 days.

Aspect 18: The method of Aspect 17, wherein the mean time between failures is greater than or equal to 18 months, preferably greater than or equal to 24 months, or greater than or equal to 36 months.

Aspect 19: Use of the rotary vacuum filter of any one or more of Aspects 1 to 14 or the filter unit of any one or more of Aspects 15 to 16 to separate bisphenol A-phenol adduct crystals from a crystallization liquor.

Aspect 20: The rotary vacuum filter of any one or more of Aspects 1 to 14 or the filter unit of any one or more of Aspects 15 to 16, wherein an average weight change of the crystallization liquor during separation of the bisphenol A-phenol adduct crystals from the crystallization liquor by said filter cloth is equal to or greater than 4.0 grams per second.

Aspect 21: Use of a non-polymeric filter cloth to separate bisphenol A-phenol adduct crystals from a crystallization liquor.

Aspect 22. Use of a non-polymer filter cloth according to Aspect 21, wherein the filter cloth is attached to and covering a perforated sector of a filter drum for a rotary vacuum filter.

Aspect 23: A method for attaching a filter cloth to a filter drum of a rotary vacuum filter for separating bisphenol A-phenol adduct crystals from a crystallization liquor comprising: introducing a portion of the filter cloth in a groove in the filter drum; and introducing an attaching piece adjacent to and abutting said portion of said filter cloth opposite an interior surface of said groove, wherein said attaching piece comprises an insert, a connector (e.g., bolting piece; such as a bolt, screw, rivet, or bar; preferably a screw) introduced into a portion of said inserts, or both.

Aspect 24: The filter unit of any one or more of Aspects 15 to 16, wherein a Mohs hardness of the scraper is less than the Mohs hardness of the filter cloth.

Aspect 25: The rotary vacuum filter of any one or more of Aspects 1 to 14 or Aspect 20, or the filter unit of any one or more of Aspects 15 to 16 or Aspect 24, further comprising a polymeric filter cloth adjacent to said non-polymeric filter cloth.

Aspect 26: The rotary vacuum filter of any one or more of Aspect 25, wherein said polymeric filter cloth comprises a polymer selected from polyether ether ketone, polypropylene, and combinations comprising at least one of the foregoing.

Aspect 27: A rotary vacuum filter for separation of bisphenol A-phenol adduct crystals from a crystallization liquor, the rotary vacuum filter comprising: a filter drum with a perforated sector on a lateral surface of the filter drum; a vacuum pump in fluid communication with an interior surface of the perforated sector; and at least one of: a non-polymeric filter cloth attached to the filter drum and covering said perforated sector; a scraper comprising a scraper section proximate said filter cloth for removing said bisphenol A-phenol adduct crystals from said filter cloth; an attaching piece comprises an insert positioned in a groove adjacent to said perforated sector, wherein at least a portion of said filter cloth is fixed in said groove by said insert abutting said portion of said filter cloth opposite an interior surface of said groove; and combinations comprising at least one of the foregoing.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments.

Unless specified to the contrary herein, all test standards are the most recent standard in effect as of the filing date of this application, or, if priority is claimed, the filing date of the earliest priority application in which the test standard appears.

The present application claims priority to EP17382350.1 filed on June 7, 2017, which is incorporated herein in its entirety.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A rotary vacuum filter for separation of bisphenol A-phenol adduct crystals from a crystallization liquor, the rotary vacuum filter comprising:
a filter drum with a perforated sector on a lateral surface of the filter drum;
a non-polymeric filter cloth attached to the filter drum and covering said perforated sector;
a first attaching piece for attaching said filter cloth to the filter drum, wherein said first attaching piece comprises an insert positioned in a groove adjacent to said perforated sector, wherein at least a portion of said filter cloth is fixed in said groove by said insert abutting said portion of said filter cloth opposite an interior surface of said groove;
a second attaching piece comprising a connector introduced into a portion of said insert; and
a vacuum pump in fluid communication with an interior surface of the perforated sector.

2. The rotary vacuum filter of claim 1, wherein said filter cloth is metallic.

3. The rotary vacuum filter of claim 1, comprising two or more non-polymeric filter cloths and two or more perforated sectors, wherein said non-polymeric filter cloths are each covering to one of said perforated sectors.

4. The rotary vacuum filter of claim 3, comprising 6 or more non-polymeric filter cloths.

5. The rotary vacuum filter of claim 1, wherein the grooves can have an inside width (W₂) that is greater than a surface width (W₁).

6. The rotary vacuum filter of claim 1, wherein, in the groove, an end of one non-polymeric filter cloth overlaps with an end of another non-polymeric filter cloth.

7. The rotary vacuum filter of claim 1, wherein said filter cloth is woven.

8. The rotary vacuum filter of claim 1, wherein a pore size of said filter cloth is 50 micrometers to 350 micrometers.

9. The rotary vacuum filter of claim 1, wherein a thickness of said filter cloth is equal to or less than 1 millimeter.

10. The rotary vacuum filter of claim 1, wherein one layer of said filter cloth is covering said perforated sector.

11. The rotary vacuum filter of claim 1, wherein a cloth weight of said filter cloth is equal to or greater than 0.40 kilograms per meter squared.

12. The rotary vacuum filter of claim 1, wherein the insert is a thermoplastic elastomer.

13. A filter unit comprising:
the rotary vacuum filter of claim 1; and
a scraper comprising a scraper section proximate said filter cloth for removing said bisphenol A-phenol adduct crystals from said filter cloth.

14. The filter unit of claim 13, wherein said scraper comprises at least one of polyether ether ketone or polytetrafluoroethylene.

15. A method for separating bisphenol A-phenol adduct crystals from a crystallization liquor, the method comprising:
feeding a crystallized stream comprising the bisphenol A-phenol adduct crystals and the crystallization liquor to the rotary vacuum filter of claim 1; and
separating the bisphenol A-phenol adduct crystals from the crystallization liquor to form a filter cake on said filter cloth and a crystallization liquor stream in said interior surface of said perforated sector.

16. (canceled)

17. A rotary vacuum filter for separation of bisphenol A-phenol adduct crystals from a crystallization liquor, the rotary vacuum filter comprising:
a filter drum with at least 2 perforated sectors on a lateral surface of the filter drum;
wherein each perforated sector is covered by at least one stainless steel filter cloth;
a first attaching piece for attaching each stainless steel filter cloth to the filter drum,
wherein said first attaching piece comprises an insert positioned in a groove adjacent to each perforated sector, wherein at least a portion of said stainless steel filter cloth is fixed in said groove by said insert abutting said portion of said stainless steel filter cloth opposite an interior surface of said groove; wherein, in the groove, an end of one stainless steel filter cloth overlaps with an end of another stainless steel filter cloth; wherein the groove has an inside width (W₂) that is greater than or equal to 1.1 times a surface width (W₁);
a second attaching piece comprising a connector introduced into a portion of said insert; and
a vacuum pump in fluid communication with an interior surface of the perforated sector.
